(19) United States
(12) Patent Application Publication
(10) Pub. No.: US 2026/0137377 A1
(43) Pub. Date: May 21, 2026

(54) BEVEL GEAR DRIVERS

(71) Applicant: K2M, INC., Leesburg, VA (US)

(72) Inventor: Joseph Michael Meyer, Terrace Park, OH (US)

(21) Appl. No.: 19/391,557

(22) Filed: Nov. 17, 2025

Related U.S. Application Data
(60) Provisional application No. 63/721,635, filed on Nov. 18, 2024.

(51) Int. Cl. A61B 17/00; A61B 90/00

(52) U.S. Cl. CPC A61B 17/00; A61B 90/03; A61B 2017/0046; A61B 2017/00477; A61B 2017/00738; A61B 2090/031

(57) ABSTRACT

Surgical drivers, systems, kits, and methods are provided that are suitable to provide off-axis torque to a surgical tool, implant, or instrument by a surgeon. In some embodiments, a driver may include a handle. The driver may also include a sleeve shaft coupled to the handle. The driver may also include a first gear shaft disposed within the sleeve shaft and have a first gear. The driver may also include an angled shaft that defines an aperture. The driver may also include a second gear shaft sized and configured to be inserted into the aperture. The second gear shaft may have a second gear configured to mesh with the first gear. The driver may also include at least one outer shaft that couples the sleeve shaft and the angled shaft.

### CROSS-REFERENCE TO RELATED APPLICATION

This application claims priority to U.S. Provisional Patent Application Ser. No. 63/721,635, filed Nov. 18, 2024, and is hereby incorporated by reference herein in its entirety.

### FIELD OF THE INVENTION

Surgical drivers, systems, kits, and methods are provided that are suitable to provide off-axis torque to a surgical tool, implant, or instrument by a surgeon.

### BACKGROUND OF THE INVENTION

Many instruments that use bevel gears to transmit off-axis torque are assembled in the same way—the distal gear is first inserted into the distal end of the instrument from the side, after which the proximal gear is inserted into the proximal end of the instrument until it meshes with the distal gear. Once the gears are meshed, the proximal gear must be constrained axially to resist the axial forces that are experienced during torque transmission. A retainer is used to constrain the proximal gear such that it minimizes the amount of play (i.e., slop) in the bevel gear set to maintain its proper function. This requires the engineer to specify tight tolerances over long distances, which poses manufacturing challenges.

Additionally, because the outer shaft of the instrument is both a single piece and angled at the distal end, the bore for the proximal gear must be a blind hole. Blind holes with large length-to-diameter ratios, which these instruments possess, can be difficult to machine and limit manufacturers to gun drilling. Also, machining the outer shaft results in a lot of wasted material. This is because the bar stock must be large enough to accommodate the angled distal tip. Accordingly, there has been a long felt need in the field for improved surgical drivers.

### SUMMARY

In some embodiments, a driver may include a handle. The driver may also include a sleeve shaft coupled to the handle. The driver may also include a first gear shaft disposed within the sleeve shaft and have a first gear. The driver may also include an angled shaft that defines an aperture. The driver may also include a second gear shaft sized and configured to be inserted into the aperture. The second gear shaft may have a second gear configured to mesh with the first gear. The driver may also include at least one outer shaft that couples the sleeve shaft and the angled shaft.

In some embodiments, a driver for transmitting off-axis torque may include a handle. The driver may include a sleeve shaft coupled to the handle. The sleeve shaft may include a cage and a retaining feature. The driver may include a first gear shaft disposed within the sleeve shaft having a first gear. The driver may include a knob housed by the cage and configured to receive a portion of the first gear shaft. The knob may be configured to receive one or more fixation devices to constrain the first gear shaft. The driver may include an angled shaft that defines an aperture. The driver may include a second gear shaft sized and configured to be inserted into the aperture. The second gear shaft may have a second gear configured to mesh with the first gear. The driver may include a proximal shaft that couples the sleeve shaft and the angled shaft.

In some embodiments, a method may include inserting a first gear shaft into a void defined by a sleeve shaft. The first gear shaft may have a first gear. The method may include coupling the sleeve shaft and the first gear shaft to a handle. The method may include constraining the first gear shaft with a knob and one or more fixation devices. The method may include inserting a second gear shaft into an aperture defined by an angled shaft. The second gear shaft may have a second gear. The method may include coupling the angled shaft to the sleeve shaft such that the first gear and the second gear mesh together. The method may include constraining the second gear shaft with at least one outer shaft.

In some embodiments, a method of assembling a driver may include inserting a first gear shaft into a void defined by a sleeve shaft. The first gear shaft may have a first gear. The method may include coupling the sleeve shaft and the first gear shaft to a handle. The method may include loading the first gear shaft with a biasing member disposed between the handle and the first gear shaft. The method may include constraining the first gear shaft with a knob and one or more fixation devices. The method may include inserting a second gear shaft into an aperture defined by an angled shaft. The second gear shaft may have a second gear. The method may include inserting a bearing between the first gear and the second gear. The method may include coupling the angled shaft to the sleeve shaft such that the first gear and the second gear are meshed. The method may include constraining the second gear shaft with at least one outer shaft.

### BRIEF DESCRIPTION OF THE DRAWINGS

The features and advantages of the invention will be more fully disclosed in, or rendered obvious by, the following detailed exemplary descriptions of embodiments. The detailed descriptions of these exemplary embodiments are to be considered together with the accompanying drawings, wherein like numbers refer to like parts and further wherein:

FIG. 1 illustrates an isometric view of a bevel gear driver in accordance with some embodiments;

FIG. 2 illustrates a side view of a bevel gear driver in accordance with some embodiments;

FIG. 3 illustrates an isometric view of a handle of a bevel gear driver in accordance with some embodiments;

FIG. 4 illustrates a cross-sectional view of a bevel gear driver along axis 4-4 illustrated in FIG. 2 in accordance with some embodiments;

FIG. 5 illustrates an isometric view of a sleeve shaft of a bevel gear driver in accordance with some embodiments;

FIG. 6 illustrates an isometric view of a first gear shaft of a bevel gear driver in accordance with some embodiments;

FIG. 7 illustrates an isometric view of a knob of a bevel gear driver in accordance with some embodiments;

FIG. 8 illustrates an isometric view of a second gear shaft of a bevel gear driver in accordance with some embodiments;

FIG. 9 illustrates an isometric view of an angled shaft of a bevel gear driver in accordance with some embodiments;

FIG. 10 illustrates an isometric view of a proximal shaft of a bevel gear driver in accordance with some embodiments;

FIG. 11 illustrates an isometric view of a distal shaft of a bevel gear driver in accordance with some embodiments;

FIG. 12 illustrates an exploded view of a bevel gear driver in accordance with some embodiments;

FIG. 13 illustrates a second isometric view of a bevel gear driver in accordance with some embodiments;

FIG. 14 illustrates a cross-sectional view of a bevel gear driver along axis 14-14 illustrated in FIG. 13;

FIG. 15 illustrates a partial cross-sectional view of a bevel gear driver along detail H illustrated in FIG. 14 in accordance with some embodiments;

FIG. 16 illustrates a partial exploded view of a bevel gear driver in accordance with some embodiments;

FIG. 17 illustrates a partial cross-sectional view of a bevel gear driver along detail G illustrated in FIG. 14 in accordance with some embodiments;

FIG. 18 illustrates a cross-sectional view of a bevel gear driver along axis 18-18 illustrated in FIG. 17 in accordance with some embodiments;

FIG. 19 illustrates a side view of a second bevel gear driver in accordance with some embodiments;

FIG. 20 illustrates a cross-sectional view of a second bevel gear driver along axis 20-20 illustrated in FIG. 19 in accordance with some embodiments;

FIG. 21 illustrates an isometric view of a first gear shaft of a second bevel gear driver in accordance with some embodiments;

FIG. 22 illustrates a first exemplary method of assembling a bevel gear driver in accordance with some embodiments; and

FIG. 23 illustrates a second exemplary method of assembling a bevel gear driver in accordance with some embodiments.

### DETAILED DESCRIPTION

This description of the exemplary embodiments is intended to be read in connection with the accompanying drawings, which are to be considered part of the entire written description. It should be understood, however, that the present disclosure is not intended to be limited to the particular forms disclosed and that the drawings are not necessarily shown to scale. Rather, the present disclosure covers all modifications, equivalents, and alternatives that fall within the spirit and scope of these exemplary embodiments. In the description, relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The terms “couple,” “coupled,” “operatively coupled,” “operatively connected,” and the like should be broadly understood to refer to connecting devices or components together either mechanically, or otherwise, such that the connection allows the pertinent devices or components to operate with each other as intended by virtue of that relationship.

The drivers, systems, kits, and methods being disclosed allow a surgeon to transmit off-axis torque to a surgical tool, instrument, or implant during a procedure. The drivers, systems, kits, and methods being disclosed also provide for assembly of a bevel gear set from a distal end of the driver. Some advantages of the drivers disclosed herein includes the reduction of the length-to-diameter ratio for the blind hole, or slot, used in the assembly of the driver. This allows the engineer to specify looser tolerances and slop in the bevel gear set is greatly reduced.

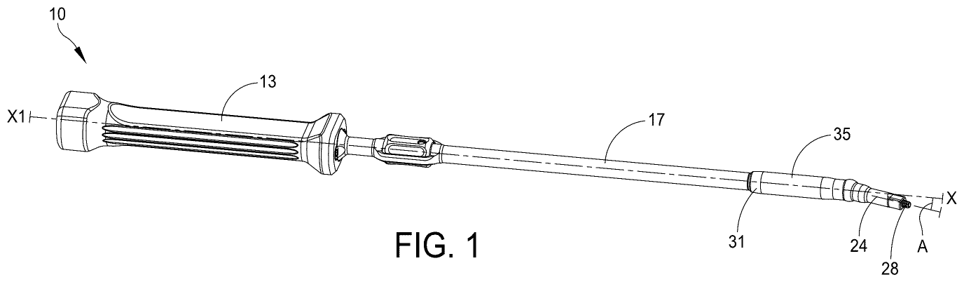

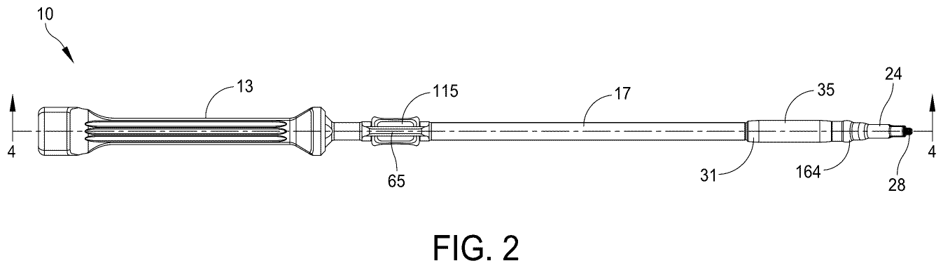

Turning now to the drawings, FIGS. 1-2 illustrate isometric and side views of a bevel gear driver 10 in accordance with some embodiments. Bevel gear driver 10 has a handle 13, a sleeve shaft 17, a first gear shaft 21 (as best illustrated in FIG. 6), an angled shaft 24, a second gear shaft 28, and at least one outer shaft, such as a proximal shaft 31 and a distal shaft 35.

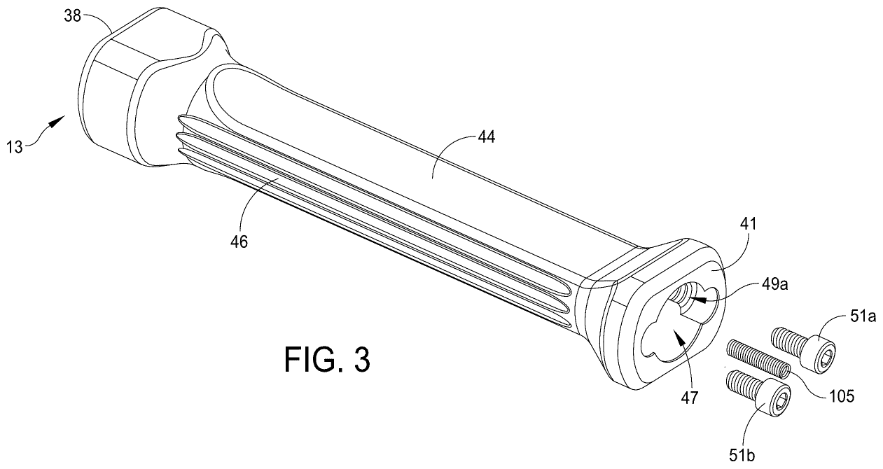

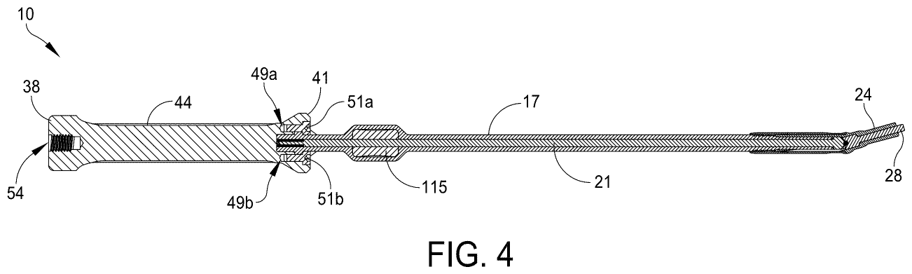

FIG. 3 illustrates an isometric view of handle 13 of bevel gear driver 10 and FIG. 4 illustrates a cross-sectional view of bevel gear driver 10 along axis 4-4 illustrated in FIG. 2 in accordance with some embodiments. Handle 13 extends between a first end 38 and a second end 41. Handle 13 defines a grip 44 disposed between first end 38 and second end 41. Grip 44 is sized and configured to be grasped by a surgeon. In some embodiments, grip 44 includes a grip feature 46, such as a non-skid surface, a plurality of ridges, a wrap, etc. to better facilitate grasping handle 13 by a surgeon.

Second end 41 of handle 13 defines a void 47 and one or more apertures 49a-b. Void 47 is sized and configured to receive sleeve shaft 17 and first gear shaft 21 as discussed in more detail below. Apertures 49a-b are sized and configured to receive respective fasteners 51a-b. Fasteners 51a-b may be a nail, screw, pin, clip, or some other suitable fasteners. For example, apertures 49a-b may be threaded and fasteners 51a-b are threaded screws configured to be removably coupled to the threaded apertures 49a-b. In some embodiments, first end 38 of handle defines a hole 54 sized and configured to receive a surgical tool or instrument. Hole 54 may be threaded and configured to receive a threaded surface of a surgical tool or instrument, such as a slap hammer.

Handle 13 may be any suitable material, such as a metal, metal alloy, or plastic. In some embodiments, handle 13 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, handle 13 may be formed from other materials, including metals, ceramics, and other materials that are suitable for use in surgery as will be understood by one of ordinary skill in the art. In some embodiments, handle 13 may be machined and/or formed using an additive manufacturing process, such as electron beam melting (EBM) or direct metal laser sintering (DMLS), to list only a few possibilities.

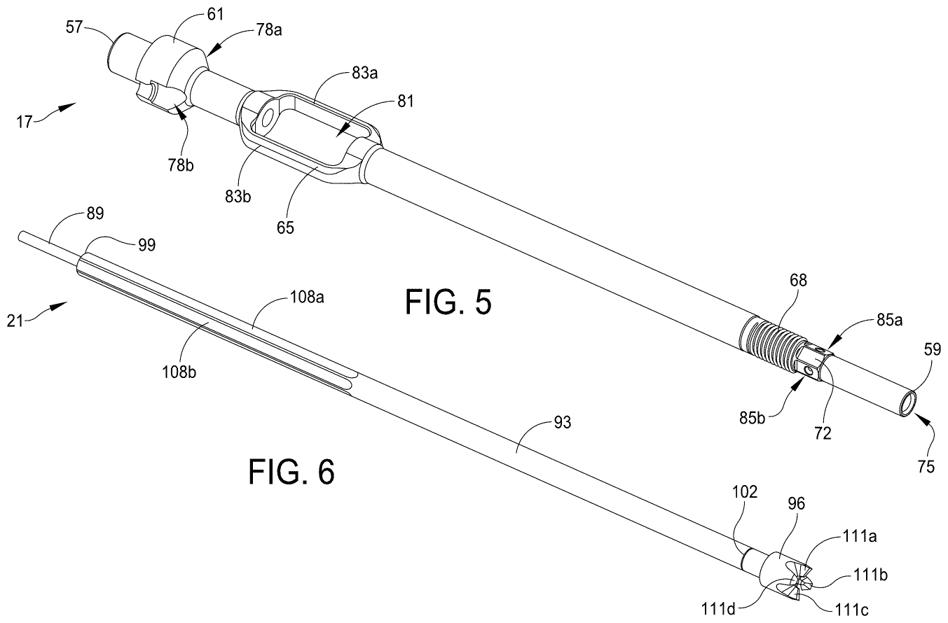

FIG. 5 illustrates an isometric view of sleeve shaft 17 of bevel gear driver 10 in accordance with some embodiments. Sleeve shaft 17 extends between a first end 57 and a second end 59. Sleeve shaft 17 includes a fixation member 61, a cage 65, a securing portion 68, and a retaining feature 72. Sleeve shaft 17 defines a void 75 along the length of sleeve shaft 17 that is sized and configured to receive first gear shaft 21. Fixation member 61 defines one or more apertures 78a-b near first end 57. Apertures 78a-b are sized and configured to receive fasteners 51a-b to couple sleeve shaft 17 to handle 13. In some embodiments, sleeve shaft 17 defines a slot 81 such that cage 65 is defined around slot 81. Cage 65 comprises one or more bars 83a-b that extend away from sleeve shaft 17.

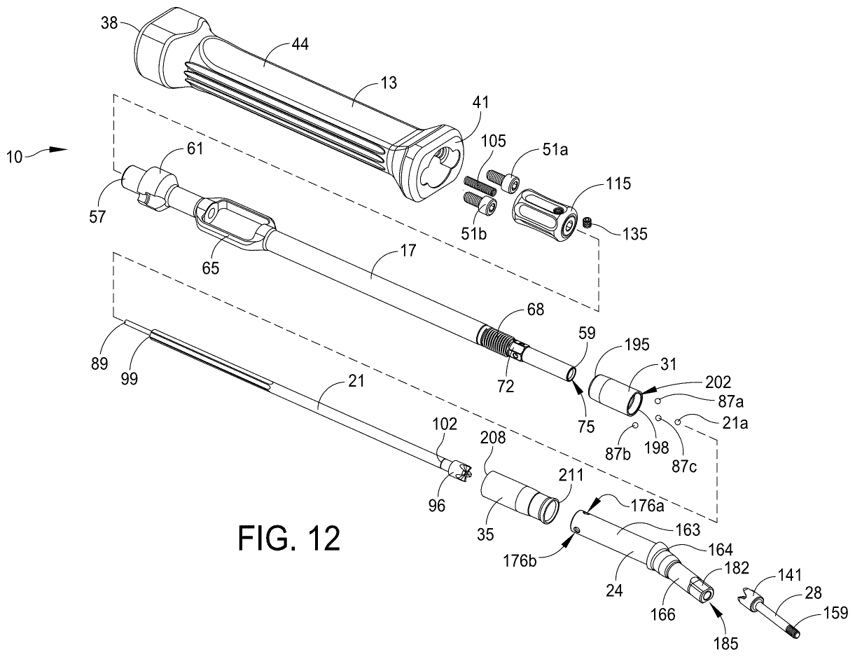

Securing portion 68 includes one or more securing features, such as a threaded portion, sized and configured to couple the at least one outer shaft (e.g., proximal shaft 31 and/or distal shaft 35) to sleeve shaft 17. For example, securing portion 68 may have threads that are configured to engage respective threads on the at least one outer shaft. Retaining feature 72 defines one or more holes 85a-b. Holes 85a-b are sized and configured to receive bearings 87a-c as best illustrated in FIG. 12 and discussed in more detail below. Retaining feature 72 may be circular, square, hexagonal, or some other polygonal shape.

Sleeve shaft 17 may be any suitable material, such as a metal, metal alloy, or plastic. In some embodiments, sleeve shaft 17 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, sleeve shaft 17 may be formed from other materials, including metals, ceramics, and other materials that are suitable for use in surgery as will be understood by one of ordinary skill in the art. In some embodiments, sleeve shaft 17 may be machined and/or formed using an additive manufacturing process, such as electron beam melting (EBM) or direct metal laser sintering (DMLS), to list only a few possibilities.

It will be appreciated that portions of sleeve shaft 17 (e.g., fixation member 61, cage 65, securing portion 68, and retaining feature 72) may be discrete parts coupled together or are formed integrally together. In some embodiments, portions of sleeve shaft 17 (e.g., fixation member 61, cage 65, securing portion 68, and retaining feature 72) are the same material. In some embodiments, portions of sleeve shaft 17 (e.g., fixation member 61, cage 65, securing portion 68, and retaining feature 72) are a different material.

FIG. 6 illustrates an isometric view of first gear shaft 21 of bevel gear driver 10 in accordance with some embodiments. First gear shaft 21 has a rod 89, a body 93, and a first gear 96. In some embodiments, first gear shaft 21 may not include rod 89. Body 93 extends between a first end 99 and a second end 102. Rod 89 may be coupled to first end 99 of body 93. Rod 89 is sized and configured to receive a biasing member 105 as best illustrated in FIG. 3. In some embodiments, biasing member 105 is a spring. Body 93 defines one or more engagement features 108a-b near first end 99. First gear 96 is coupled to second end 102 of body 93. First gear 96 defines one or more teeth 111a-d.

First gear shaft 21 may be any suitable material, such as a metal, metal alloy, or plastic. In some embodiments, first gear shaft 21 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, first gear shaft 21 may be formed from other materials, including metals, ceramics, and other materials that are suitable for use in surgery as will be understood by one of ordinary skill in the art. In some embodiments, first gear shaft 21 may be machined and/or formed using an additive manufacturing process, such as electron beam melting (EBM) or direct metal laser sintering (DMLS), to list only a few possibilities.

It will be appreciated that portions of first gear shaft 21 (e.g., rod 89, body 93, and first gear 96) may be discrete parts coupled together or are formed integrally together. In some embodiments, portions of first gear shaft 21 (e.g., rod 89, body 93, and first gear 96) are the same material. In some embodiments, portions of first gear shaft 21 (e.g., rod 89, body 93, and first gear 96) are a different material.

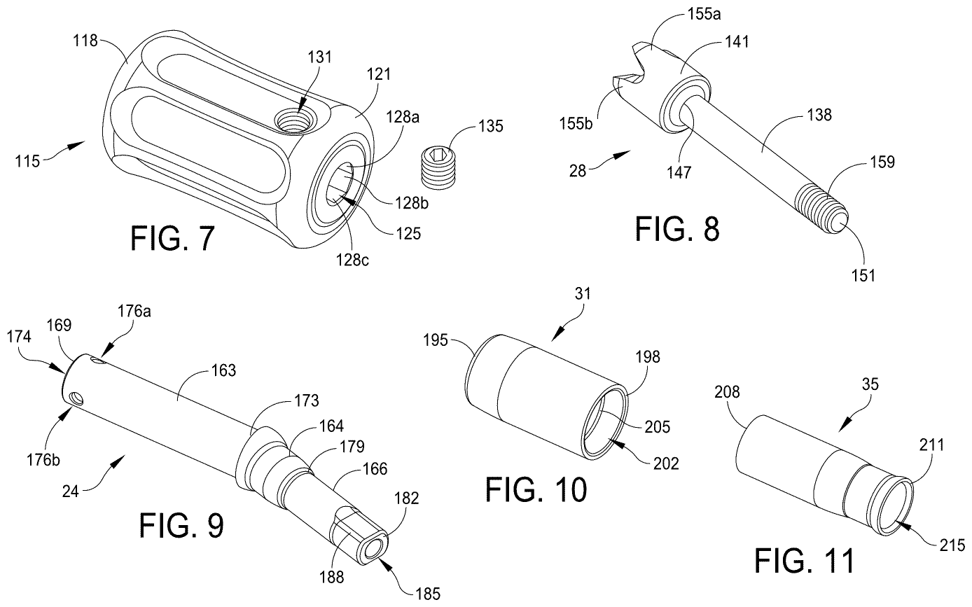

FIG. 7 illustrates an isometric view of a knob 115 of bevel gear driver 10 in accordance with some embodiments. Knob 115 extends between a first end 118 to a second end 121. Knob 115 defines a void 125 that extends between first end 118 and second end 121. Void 125 is sized and configured to receive first gear shaft 21. In some embodiments, knob 115 defines one or more engagement features 128a-c disposed within void 125. Engagement features 128a-c are configured to engage respective engagement features 108a-b of first gear shaft 21.

Knob 115 defines one or more fixation apertures 131. In some embodiments, fixation aperture 131 is threaded. Fixation aperture 131 is sized and configured to receive a fixation device 135. Fixation device 135 may be a screw, a nail, a pin, a clip, retaining ring, thrust bearing, or some other suitable fixation device. As an example, fixation aperture 131 is threaded and is configured to receive a threaded screw to secure first gear shaft 21 to knob 115 and axially constrain first gear shaft 21 when first gear shaft 21 is inserted through void 125. When first gear shaft 21 is constrained by knob 115 and fixation device 135, rotation of knob 115 rotates first gear shaft 21 and transmits torque to second gear shaft 28 as discussed in more detail below.

Knob 115 may be any suitable material, such as a metal, metal alloy, or plastic. In some embodiments, knob 115 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, knob 115 may be formed from other materials, including metals, ceramics, and other materials that are suitable for use in surgery as will be understood by one of ordinary skill in the art. In some embodiments, knob 115 may be machined and/or formed using an additive manufacturing process, such as electron beam melting (EBM) or direct metal laser sintering (DMLS), to list only a few possibilities.

Although knob 115 has been discussed as a being discrete from sleeve shaft 17, it will be appreciated that knob 115 may be formed integrally with one or more parts of sleeve shaft 17. For example, knob 115 may be coupled to or formed with cage 65. In some embodiments, knob 115 may not be needed and a dummy knob may be used as the input of torque to the second gear shaft 28.

FIG. 8 illustrates an isometric view of second gear shaft 28 of bevel gear driver 10 in accordance with some embodiments. Second gear shaft 28 has a body 138 and a second gear 141. Body 138 extends between a first end 147 and a second end 151. Second gear 141 is coupled to first end 147 of body 138. Second gear 141 defines one or more teeth 155a-b. Teeth 155a-b are sized and configured to mesh with teeth 111a-d of first gear 96. Second end 151 of body 138 defines a tip 159. Tip 159 is sized and configured to receive a surgical tool, instrument, or implant. In some embodiments, tip 159 is threaded and configured to receive respective threads of a surgical tool, instrument, or implant. In other embodiments, tip 159 defines an aperture configured to receive a surgical tool, instrument, or implant. For example, tip 159 may be sized and configured to receive a drill bit or fastener head (e.g., flat head bit, Philips-head bit, Allen-drive bit, etc.). In further embodiments, tip 159 may be formed integrally with a surgical tool or instrument such that the surgical tool or instrument is disposed at the second end 151 of body 138.

Second gear shaft 28 may be any suitable material, such as a metal, metal alloy, or plastic. In some embodiments, second gear shaft 28 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, second gear shaft 28 may be formed from other materials, including metals, ceramics, and other materials that are suitable for use in surgery as will be understood by one of ordinary skill in the art. In some embodiments, second gear shaft 28 may be machined and/or formed using an additive manufacturing process, such as electron beam melting (EBM) or direct metal laser sintering (DMLS), to list only a few possibilities.

It will be appreciated that body 138 and second gear 141 may be discrete parts coupled together or are formed integrally together. In some embodiments, body 138 and second gear 141 are the same material. In some embodiments, body 138 and second gear 141 are a different material.

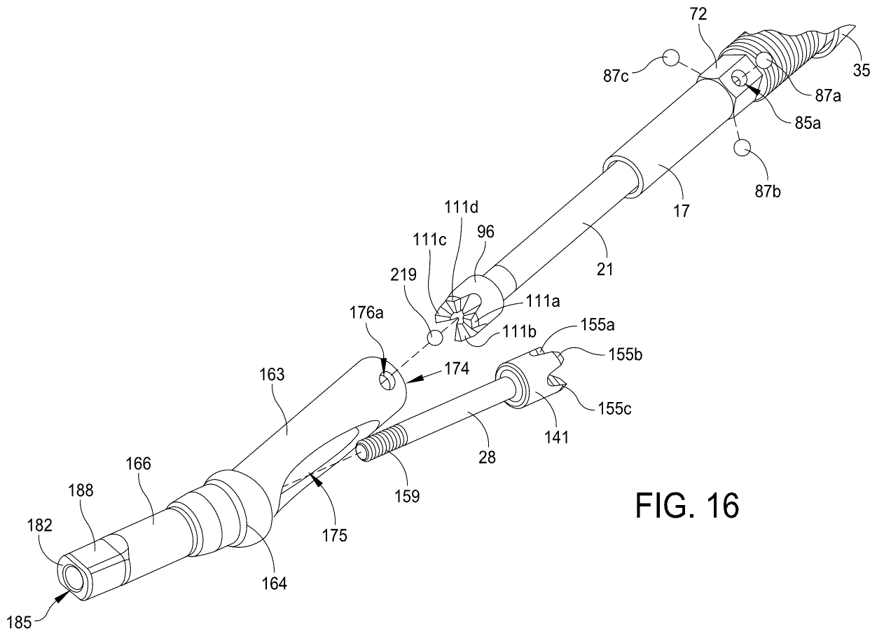

FIG. 9 illustrates an isometric view of angled shaft 24 of bevel gear driver 10 in accordance with some embodiments. Angled shaft 24 has a first portion 163, a joint 164, and a second portion 166. First portion 163 extends between a first end 169 and a second end 173. First portion 163 defines a void 174 that extends between first end 169 and second end 173. First portion 163 defines an aperture 175 as best illustrated in FIG. 16. Aperture 175 is sized and configured to receive second gear shaft 28. First portion 163 also defines one or more slots 176a-b. Slots 176a-b are sized and configured to receive a respective bearing (e.g., bearings 87a-c) as discussed in more detail below.

Second portion 166 extends between a first end 179 and a second end 182. Second portion 166 defines a void 185 that extends between first end 179 and second end 182. Second end 182 of second portion 166 defines a seat 188. Seat 188 is sized and configured to receive a portion of a surgical tool, instrument, or implant.

First portion 163 and second portion 166 are coupled together at joint 164. In some embodiments, first portion 163 and second portion 166 are removably or fixedly coupled to joint 164. In some embodiments, first portion 163 and second portion 166 are formed integrally with joint 164. Void 174 of first portion 163 and void 185 of second portion 166 are connected such that angled shaft 24 is configured to receive second gear shaft 28 therein. For example, second gear shaft 28 may be inserted into aperture 175 of first portion 163 and positioned such that second gear 141 is disposed within joint 164 and tip 159 extends out of void 185 of second portion 166.

Angled shaft 24 may be any suitable material, such as a metal, metal alloy, or plastic. In some embodiments, angled shaft 24 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, angled shaft 24 may be formed from other materials, including metals, ceramics, and other materials that are suitable for use in surgery as will be understood by one of ordinary skill in the art. In some embodiments, angled shaft 24 may be machined and/or formed using an additive manufacturing process, such as electron beam melting (EBM) or direct metal laser sintering (DMLS), to list only a few possibilities.

It will be appreciated that portions of angled shaft 24 (e.g., first portion 163, joint 164, and second portion 166) may be discrete parts coupled together or are formed integrally together. In some embodiments, portions of angled shaft 24 (e.g., first portion 163, joint 164, and second portion 166) are the same material. In some embodiments, portions of angled shaft 24 (e.g., first portion 163, joint 164, and second portion 166) are a different material.

FIG. 10 illustrates an isometric view of proximal shaft 31 of bevel gear driver 10 in accordance with some embodiments. Proximal shaft 31 extends between a first end 195 and a second end 198. Proximal shaft 31 defines a void 202 that extends between first end 195 and second end 198. In some embodiments, proximal shaft 31 defines a securing feature 205 disposed within void 202. For example, securing feature 205 of proximal shaft 31 may be threaded and configured to removably engage with threaded securing portion 68 of sleeve shaft 17.

Proximal shaft 31 may be any suitable material, such as a metal, metal alloy, or plastic. In some embodiments, proximal shaft 31 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, proximal shaft 31 may be formed from other materials, including metals, ceramics, and other materials that are suitable for use in surgery as will be understood by one of ordinary skill in the art. In some embodiments, proximal shaft 31 may be machined and/or formed using an additive manufacturing process, such as electron beam melting (EBM) or direct metal laser sintering (DMLS), to list only a few possibilities.

FIG. 11 illustrates an isometric view of distal shaft 35 of bevel gear driver 10 in accordance with some embodiments. Distal shaft 35 extends between a first end 208 and a second end 211. Distal shaft 35 defines a void 215 that extends between first end 208 and second end 211. Void 215 is sized and configured to receive first portion 163 of angled shaft 24.

Distal shaft 35 may be any suitable material, such as a metal, metal alloy, or plastic. In some embodiments, distal shaft 35 may be formed from a medical-grade material that is capable of being 3D printed (e.g., additively manufactured), such as ABS (acrylonitrile butadiene styrene), PLA (polylactic acid), PETG (polyethylene terephthalate glycol), nylon, TPU (thermoplastic polyurethane), resin, and other suitable thermoplastics and thermosetting plastics. However, distal shaft 35 may be formed from other materials, including metals, ceramics, and other materials that are suitable for use in surgery as will be understood by one of ordinary skill in the art. In some embodiments, distal shaft 35 may be machined and/or formed using an additive manufacturing process, such as electron beam melting (EBM) or direct metal laser sintering (DMLS), to list only a few possibilities.

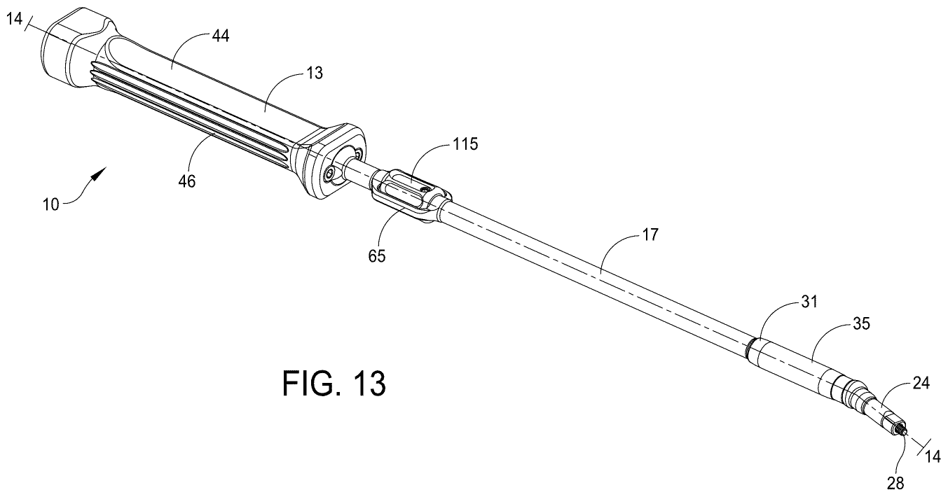

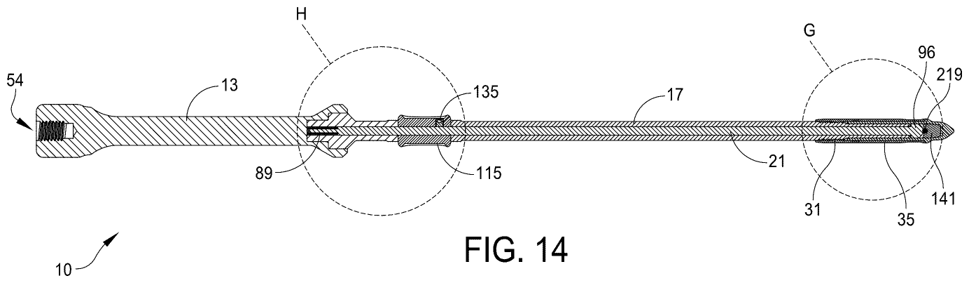

FIG. 12 illustrates an exploded view of bevel gear driver 10 in accordance with some embodiments. Bevel gear driver 10 may be sold already assembled, as illustrated in FIGS. 13 and 14, or come as a kit for a user to assemble as illustrated in FIG. 12. It will be appreciated that bevel gear driver 10 may be disassembled and reassembled by a user for cleaning, maintenance, repair, or to exchange one or more parts. For example, a user may desire a second gear shaft 28 with a different tip 159 as discussed herein.

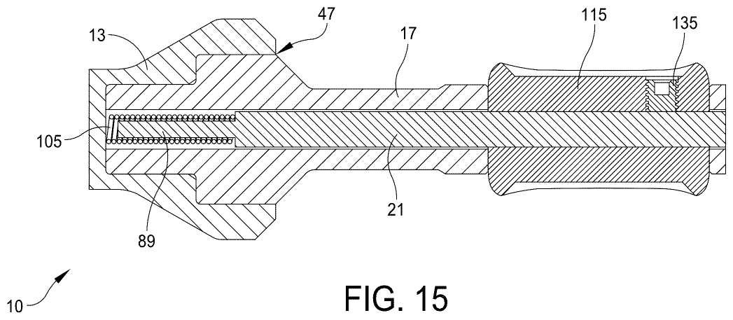

FIG. 15 illustrates a partial cross-sectional view of bevel gear driver 10 along detail H illustrated in FIG. 14 in accordance with some embodiments. Bevel gear driver 10 is assembled by inserting first gear shaft 21 through void 75 of sleeve shaft 17 from the second end 59 to first end 57 as best illustrated in FIG. 5. First gear shaft 21 is also inserted through void 125 of knob 115 from second end 121 to first end 118 as best illustrated in FIG. 7. Fixation device 135 is then inserted into fixation aperture 131 to engage first gear shaft 21 to couple knob 115 to first gear shaft 21. In some embodiments, biasing member 105 is engaged with rod 89 of first gear shaft 21 and handle 13 when the assemble sleeve shaft 17 and first gear shaft 21 are inserted into void 47 of handle 13. For example, when biasing member 105 is a spring, it may be placed over the rod 89 and engage handle 13 and first gear shaft 21 such that first gear shaft 21 is biased, or “loaded” against handle 13.

FIG. 16 illustrates a partial exploded view of bevel gear driver 10 in accordance with some embodiments. With handle 13, sleeve shaft 17, and first gear shaft 21 assembled as discussed above, angled shaft 24 and second gear shaft 28 can be assembled to bevel gear driver 10. Proximal shaft 31 and distal shaft 35 are placed over first gear shaft 21 and sleeve shaft 17 such that first gear shaft 21 and sleeve shaft 17 are inserted through void 202 of proximal shaft 31 and void 215 of distal shaft 35. During assembly of angled shaft 24 and second gear shaft 28 to first gear shaft 21 and sleeve shaft 17, proximal shaft 31 and distal shaft 35 are moved up toward handle 13.

Second gear shaft 28 is inserted through void 174 or aperture 175 such that tip 159 protrudes through void 185 of second end 182 of second portion 166. Assembled second gear shaft 28 and angled shaft 24 may then be assembled with first gear shaft 21 and sleeve shaft 17 by inserting first gear shaft 21 through void 174 such that teeth 111a-d of first gear 96 and respective teeth 155a-c of second gear 141 mesh together. In some embodiments, a bearing 219 may be placed between first gear 96 and second gear 141 to help transfer torque from first gear 96 to second gear 141 when tip 159 is off-axis (i.e., at angle A from axis X of bevel gear driver 10 as illustrated in FIG. 1).

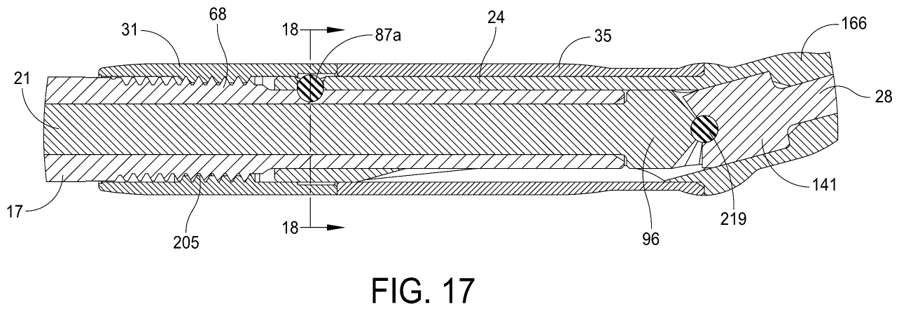

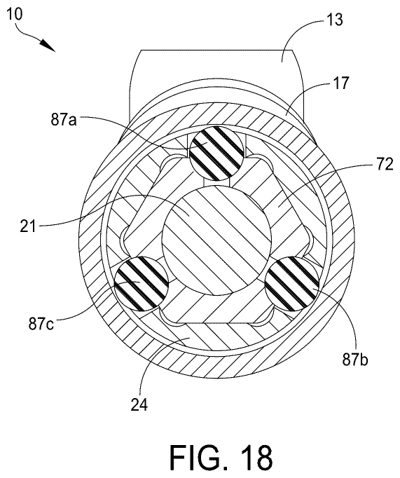

FIG. 17 illustrates a partial cross-sectional view of bevel gear driver 10 along detail G illustrated in FIG. 14 and FIG. 18 illustrates a cross-sectional view of bevel gear driver 10 along axis 18-18 illustrated in FIG. 17 in accordance with some embodiments. Angled shaft 24 is coupled to sleeve shaft 17 by inserting bearings 87a-c into respective holes 85a-c of retaining feature 72 and through respective slots 176a-c of angled shaft 24. Distal shaft 35 and proximal shaft 31 are then slid down over sleeve shaft 17 and angled shaft 24 such that distal shaft 35 covers aperture 175 and proximal shaft 31 constrains bearings 87a-c. Securing feature 205 of proximal shaft 31 engages securing portion 68 of sleeve shaft 17 to lock angled shaft 24 and second gear shaft 28 to first gear shaft 21 and sleeve shaft 17. For example, a threaded securing feature 205 may engage a threaded securing portion 68 to removably couple angled shaft 24 and second gear shaft 28 to first gear shaft 21 and sleeve shaft 17.

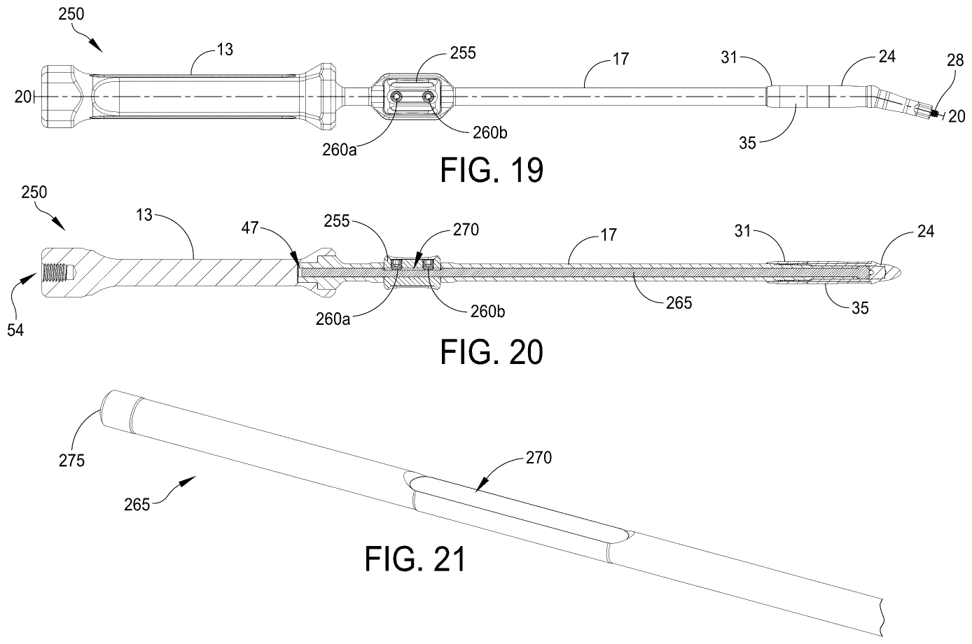

FIG. 19 illustrates a side view of a second bevel gear driver 250 in accordance with some embodiments. Bevel gear driver 250 is similar to bevel gear driver 10 discussed above and thus similar disclosure is not repeated herein. Bevel gear driver 250 has a handle 13, a sleeve shaft 17, a first gear shaft 265 (as best illustrated in FIGS. 20-21), an angled shaft 24, a second gear shaft 28, and at least one outer shaft, such as proximal shaft 31 and a distal shaft 35.

FIG. 20 illustrates a cross-sectional view of bevel gear driver 250 along axis 20-20 illustrated in FIG. 19 in accordance with some embodiments. Bevel gear driver 250 has a knob 255. Knob 255 is similar to knob 115 discussed above and thus similar disclosure is not repeated herein. Knob 255 defines one or more fixation apertures 131a-b (i.e., two fixation apertures 131 as discussed above and illustrated in FIG. 7). Fixation apertures 131a-b are sized and configured to receive a respective fixation device 260a-b. Fixation devices 260a-b may be a screw, a nail, a pin, a clip, retaining ring, thrust bearing, or some other suitable fixation device. As an example, fixation apertures 131a-b are threaded and are configured to receive a respective threaded screw to secure first gear shaft 265 to knob 255 and axially constrain first gear shaft 265 when first gear shaft 265 is inserted into knob 255. When first gear shaft 265 is constrained by knob 255 and fixation devices 260a-b, rotation of knob 255 rotates first gear shaft 265 and transmits torque to second gear shaft 28 as discussed in more detail above. It will be appreciated that knob 255 may not include engagement features, such as engagement features 128a-c discussed above regarding knob 115.

FIG. 21 illustrates an isometric view of first gear shaft 265 of bevel gear driver 250 in accordance with some embodiments. First gear shaft 265 is similar to first gear shaft 21 discussed above and thus similar disclosure is not repeated herein. First gear shaft 265 defines an engagement feature 270. Engagement feature 270 is sized and configured to receive fixation devices 260a-b to couple first gear shaft 265 to knob 255. In some embodiments, first gear shaft 265 may not include a rod coupled to a first end 275 as discussed above with reference to first gear shaft 21 (e.g., rod 89). As such, bevel gear driver 250 may not include a biasing member, such as biasing member 105 discussed above, to bias or “load” first gear shaft 265 against handle 13.

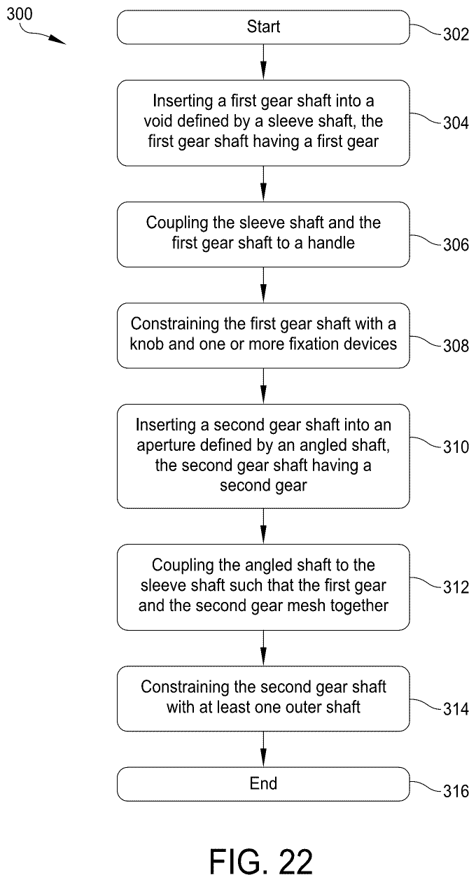

FIG. 22 illustrates a first exemplary method 300 of assembling bevel gear driver 10, 250 in accordance with some embodiments. Method 300 starts at block 302. At block 304, method 300 comprises inserting a first gear shaft 21, 265 into a void 75 defined by a sleeve shaft 17, the first gear shaft 21, 265 having a first gear 96. At block 306, method 300 comprises coupling the sleeve shaft 17 and the first gear shaft 21, 265 to a handle 13. At block 308, method 300 comprises constraining the first gear shaft 21, 265 with a knob 115, 255 and one or more fixation devices 135, 260a-b. At block 310, method 300 comprises inserting a second gear shaft 28 into an aperture 175 defined by an angled shaft 24, the second gear shaft 28 having a second gear 141. At block 312, method 300 comprises coupling the angled shaft 24 to the sleeve shaft 17 such that the first gear 96 and the second gear 141 mesh together. At block 314, method 300 comprises constraining the second gear shaft 28 with at least one outer shaft (e.g., proximal shaft 31 and/or distal shaft 35). Method 300 ends at block 316.

In some embodiments, the coupling of the sleeve shaft 17 to the handle 13 is facilitated by one or more fasteners 51a-b. In some embodiments, method 300 includes loading the first gear shaft 21 with a biasing member 105 disposed between the handle 13 and the first gear shaft 21. In some embodiments, method 300 further includes inserting a bearing 219 between the first gear 96 and the second gear 141. In some embodiments, the coupling of the angled shaft 24 to the sleeve shaft 17 is facilitated by a plurality of bearings 87a-c. In some embodiments, the constraining of the second gear shaft 28 comprises installing a proximal shaft 31 to the sleeve shaft 17 such that the angled shaft 24 is constrained. In some embodiments, the constraining of the second gear shaft 28 comprises installing a distal shaft 35 over the aperture 175 of the angled shaft 24 such that the second gear shaft 28 is constrained.

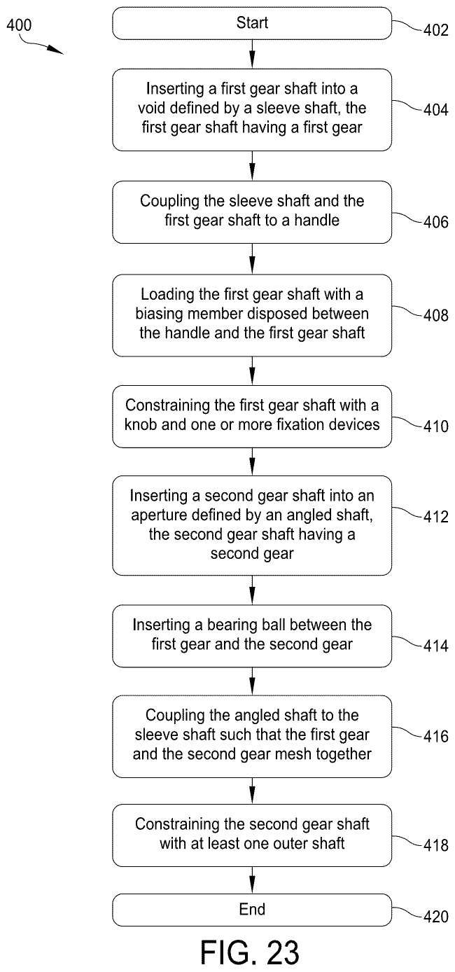

FIG. 23 illustrates a second exemplary method 400 of assembling bevel gear driver 10 in accordance with some embodiments. Method 400 starts at block 402. At block 404, method 400 comprises inserting a first gear shaft 21 into a void 75 defined by a sleeve shaft 17, the first gear shaft 21 having a first gear 96. At block 406, method 400 comprises coupling the sleeve shaft 17 and the first gear shaft 21 to a handle 13. At block 408, method 400 comprises loading the first gear shaft 21 with a biasing member 105 disposed between the handle 13 and the first gear shaft 21. At block 410, method 400 comprises constraining the first gear shaft 21 with a knob 115 and one or more fixation devices 135. At block 412, method 400 comprises inserting a second gear shaft 28 into an aperture 175 defined by an angled shaft 24, the second gear shaft 28 having a second gear 141. At block 414, method 400 comprises inserting a bearing 219 between the first gear 96 and the second gear 141. At block 416, method 400 comprises coupling the angled shaft 24 to the sleeve shaft 17 such that the first gear 96 and the second gear 141 mesh together. At block 418, method 400 comprises constraining the second gear shaft 28 with at least one outer shaft (e.g., proximal shaft 31 and/or distal shaft 35). Method 400 ends at block 420.

In some embodiments, the coupling of the angled shaft 24 to the sleeve shaft 17 is facilitated by a plurality of bearings 87a-c. In some embodiments, the constraining of the second gear shaft 28 comprises installing a proximal shaft 31 to the sleeve shaft 17 such that the angled shaft 24 is constrained. In some embodiments, the constraining of the second gear shaft 28 comprises installing a distal shaft 35 over the aperture 175 of the angled shaft 24 such that the second gear shaft 28 is constrained.

When assembled, bevel gear drivers 10, 250 may be used to transmit off-axis torque to a surgical tool, instrument, or implant. For example, bevel gear drivers 10, 250 may be used to insert a trial into a patient that is off-axis from a midline (e.g., at angle A from axis X illustrated in FIG. 1). Referring back to FIGS. 13, 17, and 19-20, a surgeon may insert a portion of bevel gear driver 10, 250, such as angled shaft 24 and second gear shaft 28, into a patient. Tip 159 of second gear shaft 28 may be connected to an implant, such as a trial, prior to insertion or may be configured to engage an implant already placed in a patient. Rotation of knob 115, 255 transmits torque to second gear 141 through rotation of first gear shaft 21, 265 and first gear 96. When angled shaft 24 is positioned at some angle A from axis X of bevel gear driver 10 (as illustrated in FIG. 1), torque is transmitted from first gear shaft 21, 265 and first gear 96 to second gear shaft 28 and second gear 141 at angle A.

### Features of the Disclosure

In some embodiments, a driver may include a handle. The driver may also include a sleeve shaft coupled to the handle. The driver may also include a first gear shaft disposed within the sleeve shaft and have a first gear. The driver may also include an angled shaft that defines an aperture. The driver may also include a second gear shaft sized and configured to be inserted into the aperture. The second gear shaft may have a second gear configured to mesh with the first gear. The driver may also include at least one outer shaft that couples the sleeve shaft and the angled shaft.

In some embodiments, the handle may extend between a first end and a second end, and may define a grip between the first end and the second end. In some embodiments, the grip may include at least one of a non-skid surface, a plurality of ridges, or a wrap. In some embodiments, the grip may be a different material than the first end and the second end. In some embodiments, the driver may include a biasing member to bias the first gear shaft against the handle.

In some embodiments, a first end of the sleeve shaft may be coupled to the handle with one or more fasteners. In some embodiments, a second end of the sleeve shaft may include a securing portion. In some embodiments, the driver may include a knob that surrounds a portion of the first gear shaft. In some embodiments, the knob may define one or more fixation apertures sized and configured to receive a respective fixation device to secure the first gear shaft to the knob. In some embodiments, the sleeve shaft may include a cage that surrounds the knob. In some embodiments, the sleeve shaft may include a retaining feature. In some embodiments, the retaining feature may be a hexagonal shape. In some embodiments, the retaining feature may define a plurality of holes each sized and configured to receive a respective bearing.

In some embodiments, the angled shaft may define a plurality of slots each sized to receive a respective bearing such that the angled shaft and the sleeve shaft are configured to be coupled together with the bearings. In some embodiments, the at least one outer shaft may have a securing feature configured to engage a securing portion of the sleeve shaft such that the at least one outer shaft constrains the bearings. In some embodiments, the at least one outer shaft may be a proximal shaft and a distal shaft. In some embodiments, the proximal shaft may couple the sleeve shaft and the angled shaft, and the distal shaft may constrain the second gear shaft. In some embodiments, the driver may include a bearing disposed between the first gear and the second gear. In some embodiments, the second gear shaft may define the second gear at a first end and a tip at a second end. In some embodiments, the tip may be threaded.

In some embodiments, a driver for transmitting off-axis torque may include a handle. The driver may include a sleeve shaft coupled to the handle. The sleeve shaft may include a cage and a retaining feature. The driver may include a first gear shaft disposed within the sleeve shaft having a first gear. The driver may include a knob housed by the cage and configured to receive a portion of the first gear shaft. The knob may be configured to receive one or more fixation devices to constrain the first gear shaft. The driver may include an angled shaft that defines an aperture. The driver may include a second gear shaft sized and configured to be inserted into the aperture. The second gear shaft may have a second gear configured to mesh with the first gear. The driver may include a proximal shaft that couples the sleeve shaft and the angled shaft.

In some embodiments, the handle may extend between a first end and a second end, and may define a grip between the first end and the second end. In some embodiments, the grip may have at least one of a non-skid surface, a plurality of ridges, or a wrap. In some embodiments, the grip may be a different material than the first end and the second end.

In some embodiments, a first end of the sleeve shaft may be coupled to the handle with one or more fasteners. In some embodiments, a second end of the sleeve shaft may include a securing portion. In some embodiments, the sleeve shaft may include a retaining feature. In some embodiments, the retaining feature may be a hexagonal shape. In some embodiments, the retaining feature may define a plurality of holes each sized to receive a respective bearing. In some embodiments, the angled shaft may define a plurality of slots each sized to receive a respective one of the plurality of bearings such that the angled shaft and the sleeve shaft are configured to be coupled together with the plurality of bearings.

In some embodiments, the proximal shaft may have a threaded securing feature configured to engage a threaded securing portion of the sleeve shaft such that the proximal shaft constrains the bearings. In some embodiments, the driver may include a distal shaft configured to constrain the second gear shaft. In some embodiments, the driver may include a bearing disposed between the first gear and the second gear. In some embodiments, the second gear shaft may define the second gear at a first end and a tip at a second end. In some embodiments, the tip may be threaded. In some embodiments, the driver may include a biasing member disposed between the first gear shaft and the handle to bias the first gear against the handle.

In some embodiments, a method may include inserting a first gear shaft into a void defined by a sleeve shaft. The first gear shaft may have a first gear. The method may include coupling the sleeve shaft and the first gear shaft to a handle. The method may include constraining the first gear shaft with a knob and one or more fixation devices. The method may include inserting a second gear shaft into an aperture defined by an angled shaft. The second gear shaft may have a second gear. The method may include coupling the angled shaft to the sleeve shaft such that the first gear and the second gear mesh together. The method may include constraining the second gear shaft with at least one outer shaft.

In some embodiments, the coupling of the sleeve shaft to the handle is facilitated by one or more fasteners. In some embodiments, the method may include loading the first gear shaft with a biasing member disposed between the handle and the first gear shaft. In some embodiments, the sleeve shaft may define a cage configured to house the knob. In some embodiments, the method may include inserting a bearing between the first gear and the second gear. In some embodiments, the coupling of the angled shaft to the sleeve shaft is facilitated by a plurality of bearings. In some embodiments, the constraining of the second gear shaft may include installing a proximal shaft to the sleeve shaft such that the angled shaft is constrained. In some embodiments, the constraining of the second gear shaft may include installing a distal shaft over the aperture of the angled shaft such that the second gear shaft is constrained.

In some embodiments, a method of assembling a driver may include inserting a first gear shaft into a void defined by a sleeve shaft. The first gear shaft may have a first gear. The method may include coupling the sleeve shaft and the first gear shaft to a handle. The method may include loading the first gear shaft with a biasing member disposed between the handle and the first gear shaft. The method may include constraining the first gear shaft with a knob and one or more fixation devices. The method may include inserting a second gear shaft into an aperture defined by an angled shaft. The second gear shaft may have a second gear. The method may include inserting a bearing between the first gear and the second gear. The method may include coupling the angled shaft to the sleeve shaft such that the first gear and the second gear are meshed. The method may include constraining the second gear shaft with at least one outer shaft.

In some embodiments, the coupling of the sleeve shaft to the handle may be facilitated by one or more fasteners. In some embodiments, the sleeve shaft may define a cage configured to house the knob. In some embodiments, the coupling of the angled shaft to the sleeve shaft is facilitated by a plurality of bearings. In some embodiments, the constraining of the second gear shaft may include installing a proximal shaft to the sleeve shaft such that the angled shaft is constrained. In some embodiments, the constraining of the second gear shaft may include installing a distal shaft over the aperture of the angled shaft such that the second gear shaft is constrained.

Although the drivers, systems, kits, and methods have been described in terms of exemplary embodiments, they are not limited thereto. Rather, the appended claims should be construed broadly, to include other variants and embodiments of the drivers, systems, kits, and methods, which may be made by those skilled in the art without departing from the scope and range of equivalents.

What is claimed is:

1. A driver comprising:
a handle;
a sleeve shaft coupled to the handle;
a first gear shaft disposed within the sleeve shaft and having a first gear;
an angled shaft defining an aperture;
a second gear shaft sized and configured to be inserted into the aperture, the second gear shaft having a second gear configured to mesh with the first gear; and
at least one outer shaft that couples the sleeve shaft and the angled shaft.

2. The driver of claim 1, wherein the handle extends between a first end and a second end, and defines a grip between the first end and the second end.

3. The driver of claim 2, wherein the grip comprises at least one of a non-skid surface, a plurality of ridges, or a wrap.

4. The driver of claim 3, wherein the grip is a different material than the first end and the second end.

5. The driver of claim 1, further comprising a biasing member to bias the first gear shaft against the handle.

6. The driver of claim 1, wherein a first end of the sleeve shaft is coupled to the handle with one or more fasteners.

7. The driver of claim 1, wherein a second end of the sleeve shaft comprises a securing portion.

8. The driver of claim 1, further comprising a knob that surrounds a portion of the first gear shaft.

9. The driver of claim 8, wherein the knob defines one or more fixation apertures sized and configured to receive a respective fixation device to secure the first gear shaft to the knob.

10. The driver of claim 9, wherein the sleeve shaft comprises a cage that surrounds the knob.

11. The driver of claim 1, wherein the sleeve shaft comprises a retaining feature.

12. The driver of claim 11, wherein the retaining feature is a hexagonal shape.

13. The driver of claim 12, wherein the retaining feature defines a plurality of holes each sized and configured to receive a respective bearing.

14. The driver of claim 13, wherein the angled shaft defines a plurality of slots each sized to receive a respective bearing such that the angled shaft and the sleeve shaft are configured to be coupled together with the bearings.

15. The driver of claim 14, wherein the at least one outer shaft has a securing feature configured to engage a securing portion of the sleeve shaft such that the at least one outer shaft constrains the bearings.

16. The driver of claim 1, wherein the at least one outer shaft is a proximal shaft and a distal shaft, and wherein the proximal shaft couples the sleeve shaft and the angled shaft, and the distal shaft constrains the second gear shaft.

17. The driver of claim 1, further comprising a bearing disposed between the first gear and the second gear.

18. The driver of claim 1, wherein the second gear shaft defines the second gear at a first end and a tip at a second end.

19. The driver of claim 18, wherein the tip is threaded.

20. A driver for transmitting off-axis torque comprising:
a handle;
a sleeve shaft coupled to the handle, the sleeve shaft comprising:
a cage; and
a retaining feature;
a first gear shaft disposed within the sleeve shaft having a first gear;
a knob housed by the cage and configured to receive a portion of the first gear shaft, the knob being configured to receive one or more fixation devices to constrain the first gear shaft;
an angled shaft defining an aperture;
a second gear shaft sized and configured to be inserted into the aperture, the second gear shaft having a second gear configured to mesh with the first gear; and
a proximal shaft that couples the sleeve shaft and the angled shaft.

21. The driver of claim 20, wherein the handle extends between a first end and a second end, and defines a grip between the first end and the second end.

22. The driver of claim 21, wherein the grip comprises at least one of a non-skid surface, a plurality of ridges, or a wrap.

23. The driver of claim 22, wherein the grip is a different material than the first end and the second end.

24. The driver of claim 20, wherein a first end of the sleeve shaft is coupled to the handle with one or more fasteners.

25. The driver of claim 20, wherein a second end of the sleeve shaft comprises a securing portion.

26. The driver of claim 20, wherein the sleeve shaft comprises a retaining feature.

27. The driver of claim 26, wherein the retaining feature is a hexagonal shape.

28. The driver of claim 27, wherein the retaining feature defines a plurality of holes each sized to receive a respective bearing.

29. The driver of claim 28, wherein the angled shaft defines a plurality of slots each sized to receive a respective one of the plurality of bearings such that the angled shaft and the sleeve shaft are configured to be coupled together with the plurality of bearings.

30. The driver of claim 29, wherein the proximal shaft has a threaded securing feature configured to engage a threaded securing portion of the sleeve shaft such that the proximal shaft constrains the bearings.

31. The driver of claim 20, further comprising a distal shaft configured to constrain the second gear shaft.

32. The driver of claim 20, further comprising a bearing disposed between the first gear and the second gear.

33. The driver of claim 20, wherein the second gear shaft defines the second gear at a first end and a tip at a second end.

34. The driver of claim 33, wherein the tip is threaded.

35. The driver of claim 20, further comprising a biasing member disposed between the first gear shaft and the handle to bias the first gear against the handle.

36. A method comprising:
inserting a first gear shaft into a void defined by a sleeve shaft, the first gear shaft having a first gear;
coupling the sleeve shaft and the first gear shaft to a handle;
constraining the first gear shaft with a knob and one or more fixation devices;
inserting a second gear shaft into an aperture defined by an angled shaft, the second gear shaft having a second gear;
coupling the angled shaft to the sleeve shaft such that the first gear and the second gear mesh together; and
constraining the second gear shaft with at least one outer shaft.

37. The method of claim 36, wherein the coupling of the sleeve shaft to the handle is facilitated by one or more fasteners.

38. The method of claim 36, further comprising loading the first gear shaft with a biasing member disposed between the handle and the first gear shaft.

39. The method of claim 36, wherein the sleeve shaft defines a cage configured to house the knob.

40. The method of claim 36, further comprising inserting a bearing between the first gear and the second gear.

41. The method of claim 36, wherein the coupling of the angled shaft to the sleeve shaft is facilitated by a plurality of bearings.

42. The method of claim 36, wherein the constraining of the second gear shaft comprises:
installing a proximal shaft to the sleeve shaft such that the angled shaft is constrained; and
installing a distal shaft over the aperture of the angled shaft such that the second gear shaft is constrained.

43. A method of assembling a driver comprising:
inserting a first gear shaft into a void defined by a sleeve shaft, the first gear shaft having a first gear;
coupling the sleeve shaft and the first gear shaft to a handle;
loading the first gear shaft with a biasing member disposed between the handle and the first gear shaft;
constraining the first gear shaft with a knob and one or more fixation devices;
inserting a second gear shaft into an aperture defined by an angled shaft, the second gear shaft having a second gear;
inserting a bearing between the first gear and the second gear;
coupling the angled shaft to the sleeve shaft such that the first gear and the second gear are meshed; and
constraining the second gear shaft with at least one outer shaft.

44. The method of claim 43, wherein the coupling of the sleeve shaft to the handle is facilitated by one or more fasteners.

45. The method of claim 43, wherein the sleeve shaft defines a cage configured to house the knob.

46. The method of claim 43, wherein the coupling of the angled shaft to the sleeve shaft is facilitated by a plurality of bearings.

47. The method of claim 43, wherein the constraining of the second gear shaft comprises:
installing a proximal shaft to the sleeve shaft such that the angled shaft is constrained; and
installing a distal shaft over the aperture of the angled shaft such that the second gear shaft is constrained.
